(19) United States
(12) Patent Application Publication
(10) Pub. No.: US 2026/0256368 A1
(43) Pub. Date: Sep. 3, 2026

(54) SENSOR MOUNT WITH EMBEDDED CONDUCTORS FOR DIFFERENT SENSOR

(71) Applicant: KONINKLIJKE PHILIPS N.V., EINDHOVEN (NL)

(72) Inventor: Eric Brett HENDERSON, ESCONDIDO, CA (US)

(21) Appl. No.: 18/993,313

(22) PCT Filed: Jul. 14, 2023
(86) PCT No.: PCT/EP2023/069623

§ 371 Date: Jan. 10, 2025

Related U.S. Application Data
(60) Provisional application No. 63/389,417, filed on Jul. 15, 2022.

(51) Int. Cl. A61B 5/0215; A61B 5/00; A61B 5/026

(52) U.S. Cl. CPC A61B 5/02158; A61B 5/026; A61B 5/6851

(57) ABSTRACT

An intraluminal device is provided. The intraluminal device includes a flexible elongate member that can be positioned within a body lumen of a patient. A sensor mount, a first sensor, and a second sensor are positioned at the distal portion of the flexible elongate member. The first sensor is positioned on the sensor mount. The second sensor is spaced from the first sensor. The sensor mount includes a conductive material that is configured to carry signals associated with the second sensor.

### TECHNICAL FIELD

The subject matter described herein relates to intraluminal physiology sensing devices (e.g., an intravascular pressure sensing and/or flow sensing guidewire). For example, the intraluminal device may include a sensor mount supporting a sensor and having embedded conductive channels for passing electrical signals to and from a different sensor.

### BACKGROUND

Existing intravascular guidewires with a sensor have fine-gauge electrical wires that provide transmission of electrical signals for the sensor. These guidewires are small and have a small diameter in order to fit inside small blood vessels. Further, the core wire and the electrical wires take up separate space inside the guidewire, and routing of electrical wires past the sensor mount of one sensor, toward a more distal second sensor, presents both design and manufacturing challenges.

The information included in this Background section of the specification, including any references cited herein and any description or discussion thereof, is included for technical reference purposes only and is not to be regarded as subject matter by which the scope of the disclosure is to be bound.

### SUMMARY

Disclosed are intraluminal physiology sensing devices (e.g., an intravascular pressure-sensing and/or flow-sensing guidewire) that include a sensor mount with embedded electrical conductive channels. This application is related to a multi-sensor intraluminal physiology sensing device (e.g., a device that includes two pressure sensors, or two flow sensors, or one pressure sensor and one flow sensor, etc.). This could also be called a combination or combo intraluminal physiology sensing device. The sensor mount for one of the sensors includes conductive materials that carries signals for another one of the sensors. That is, the sensor mount that physically supports one of the sensors has conductive material also carries the signals for a different sensor. The different sensor can be, e.g., a sensor that is not supported by the sensor mount, or that is spaced from (e.g., distal of, proximal of) the sensor mount, or that is located on a different (e.g., more distal, more proximal) portion of the sensor mount. The embedded conductive channels can allow electrical microwires or filars that are proximal of the sensor mount to be connected to a component (e.g., another sensor) located distal of the sensor mount, without the need to run filars alongside the sensor mount and directly to the distal component. Such an arrangement may significantly simplify assembly of the guidewire device, reducing both costs and the chance of manufacturing defects, while also improving the robustness of the guidewire during handling and use. In addition, this arrangement may reduce or eliminate the need to position filars between the sensor mount and a sensor housing that surrounds the sensor mount. In general, the sensor mount can physically support one sensor or a plurality of sensors, and the sensor mount can include conductive pathways to transmit signals associated with one sensor and/or a plurality of sensors.

The sensor mount with embedded conductors disclosed herein has particular, but not exclusive, utility for intraluminal medical catheters, guidewires, or guide catheters. One general aspect includes an intraluminal device. The intraluminal device includes a flexible elongate member configured to be positioned within a body lumen of a patient, wherein the flexible elongate member includes a proximal portion and a distal portion; a first sensor positioned at the distal portion of the flexible elongate member; a second sensor positioned at the distal portion of the flexible elongate member; and a sensor mount positioned at the distal portion of the flexible elongate member, wherein the first sensor is positioned on the sensor mount. The second sensor is spaced from the first sensor, and the sensor mount includes a first material that is electrically conductive and configured to carry electrical signals associated with the second sensor.

Implementations may include one or more of the following features. In some embodiments, the first sensor includes a first intraluminal modality, and the second sensor includes a different, second intraluminal modality. In some embodiments, the first sensor includes a pressure sensor, and the second sensor includes a flow sensor. In some embodiments, the sensor mount includes a second material forming an outer surface of the sensor mount, where, in a cross-section, the second material completely surrounds the conductive first material. In some embodiments, the second material is electrically conductive, the sensor mount includes a third material disposed between the first material and the second material, and the third material includes an electrically insulating material. In some embodiments, in the cross-section, the third material completely surrounds the first material. In some embodiments, the sensor mount includes a proximal portion and a distal portion, and the first material extends between the proximal portion and the distal portion. In some embodiments, the first sensor overlaps with the first material along a length of the sensor mount. In some embodiments, a majority of the first material is embedded within the sensor mount, and the first material includes a first exposed portion and a second exposed portion. In some embodiments, the intraluminal device further includes: a connector region positioned at the proximal portion of the flexible elongate member; a first electrical wire coupled to the first exposed portion and the second sensor; and a second electrical wire coupled to the second exposed portion and the connector region such that the second sensor is in electrical communication with the connector region. In some embodiments, the intraluminal device further includes: a connector region positioned at the proximal portion of the flexible elongate member; a first electrical wire coupled to the first exposed portion and the second sensor; a wire bond coupled to the second exposed portion and the first sensor; and a second electrical wire coupled to the first sensor and the connector region such that the first sensor and the second sensor is in electrical communication with the connector region. In some embodiments, the intraluminal device further includes a third electrical wire coupled to the first sensor and the connector region such that the first sensor is in electrical communication with the connector region. In some embodiments, the first exposed portion and the second exposed portion are continuous with an outer surface of the sensor mount. Implementations of the described techniques may include hardware, a method or process, or computer software on a computer-accessible medium.

One general aspect includes an apparatus. The apparatus includes an intravascular guidewire configured to be positioned within a blood vessel of a patient; a flow sensor positioned at a distal end of the intravascular guidewire; a pressure sensor positioned proximal of the flow sensor such that the pressure sensor is spaced from the distal end of the intravascular guidewire; a pressure sensor mount, wherein the pressure sensor is positioned on the pressure sensor mount; a connector region positioned at a proximal portion of the intravascular guidewire; and a flow signal pathway extending between the flow sensor and the connector region, wherein the flow signal pathway is configured to carry electrical signals associated with the flow sensor, wherein a portion of the flow signal pathway includes conductive material forming part of a structure of the pressure sensor mount. Other embodiments of this aspect include corresponding computer systems, apparatus, and computer programs recorded on one or more computer storage devices, each configured to perform the actions of the methods.

Implementations may include an apparatus further including: a pressure signal pathway extending between the pressure sensor and the connector region, and the pressure signal pathway is configured to carry electrical signals associated with the pressure sensor. Implementations of the described techniques may include hardware, a method or process, or computer software on a computer-accessible medium.

This Summary is provided to introduce a selection of concepts in a simplified form that are further described below in the Detailed Description. This Summary is not intended to identify key features or essential features of the claimed subject matter, nor is it intended to limit the scope of the claimed subject matter. A more extensive presentation of features, details, utilities, and advantages of the sensor mount with embedded conductors, as defined in the claims, is provided in the following written description of various aspects of the disclosure and illustrated in the accompanying drawings.

### BRIEF DESCRIPTION OF THE DRAWINGS

Illustrative aspects of the present disclosure will be described with reference to the accompanying drawings, of which:

FIG. 1 is a diagrammatic top view of an intravascular device, according to aspects of the present disclosure.

FIG. 2 is a diagrammatic side view of an intravascular sensing system that includes an intravascular device, according to aspects of the present disclosure.

FIG. 3 is a diagrammatic cross-sectional view of an example sensor assembly, which may for example be included in the intravascular device of FIG. 2, according to aspects of the present disclosure.

FIG. 4 is a diagrammatic cross-sectional view of an intraluminal sensing device that includes both a pressure sensor and a flow sensor, according to aspects of the present disclosure.

FIG. 5 is a diagrammatic cross-sectional side view of a sensor mount with embedded signal-carrying conductive material, according to aspects of the present disclosure.

FIG. 6 is a diagrammatic cross-sectional view of the detail region of a sensor mount with embedded signal-carrying conductive material, according to aspects of the present disclosure.

FIG. 7 is a diagrammatic top view of a sensor mount with embedded signal-carrying conductive material, according to aspects of the present disclosure.

FIG. 8 is a diagrammatic cross-sectional view of the detail region of an example sensor mount, according to aspects of the present disclosure.

FIG. 9 is a schematic view of the wiring of an intraluminal sensing device that includes both a pressure sensor and a flow sensor, according to aspects of the present disclosure.

FIG. 10 is a diagrammatic perspective view of a sensor mount whose schematic wiring diagram is shown in FIG. 9, according to aspects of the present disclosure.

FIG. 11 is a diagrammatic top view of the sensor mount of FIG. 10, according to aspects of the present disclosure.

FIG. 12 is a diagrammatic, lateral cross-sectional view of the sensor mount of FIG. 11, taken along cross-section line 12-12, according to aspects of the present disclosure.

FIG. 13 is a schematic view of the wiring of an intraluminal sensing device that includes both a pressure sensor and a flow sensor, according to aspects of the present disclosure.

FIG. 14 is a diagrammatic perspective view of a sensor mount whose schematic wiring diagram is shown in FIG. 13, according to aspects of the present disclosure.

FIG. 15 is a diagrammatic, perspective view of the detail region of the sensor mount of FIG. 14, according to aspects of the present disclosure.

FIG. 16 is a diagrammatic top view of the detail region of the sensor mount of FIG. 14, according to aspects of the present disclosure.

FIG. 17 is a schematic diagram of a processor circuit, according to at least one embodiment of the present disclosure.

### DETAILED DESCRIPTION

Disclosed are intraluminal physiology sensing devices (e.g., an intravascular pressure-sensing and/or flow-sensing guidewire or catheter) that include a sensor mount with embedded electrical conductive channels. This application is related to a multi-sensor intraluminal physiology sensing device or combination intraluminal physiology sensing device (e.g., a device that includes two pressure sensors, two flow sensors, one pressure sensor and one flow sensor, etc.). The sensor mount for a first of the sensors includes conductive materials that carries signals for another of the sensors. That is, the sensor mount that physically supports the first of the sensors has conductive material also carries the signals for a different sensor (e.g., a sensor that is not supported by the sensor mount or that is spaced from the sensor mount and/or the first sensor).

The embedded conductive channels can allow for electrical connection of microwires or filars that are proximal of the sensor mount to a component (e.g., another sensor such as a flow sensor) that are located distal of the sensor mount, without the need to run filars alongside the sensor mount and directly to the distal component. Such an arrangement may significantly simplify designs of the guidewire device, along with assembly procedures. This simplification may reduce both costs and the chance of manufacturing defects, while also improving the robustness of the intraluminal sensing device during handling and use. In addition, this arrangement may reduce or eliminate the need to position filars between the sensor mount and a sensor housing that surrounds the sensor mount. This may in turn allow for a tighter fit between the sensor mount and the sensor housing, or for a larger sensor mount, or a smaller sensor housing. A smaller sensor housing may be advantageous by, for example, allowing for a smaller outer diameter of the intraluminal sensing device. In some embodiments, this arrangement may even allow the sensor mount and sensor housing to be combined into a single component, thus further simplifying designs and manufacturing/assembly procedures.

Example devices incorporating a multi-filar conductor bundle and/or conductive ribbons include intraluminal medical guidewire devices as described for example in U.S. Pat. No. 10,595,820 B2, U.S. Patent Publication Nos. 2014/0187874, 2016/0058977, and 2015/0273187, and in U.S. Provisional Patent Application No. 62/552,993, filed Aug. 31, 2017, each of which is hereby incorporated by reference in its entirety as though fully set forth herein. Example devices incorporating both pressure sensors and flow sensors can be found for example in U.S. Pat. No. 8,231,537, which is hereby incorporated by reference in its entirety as though fully set forth herein. Examples of flow sensor housings can be found for example in U.S. Provisional Patent Application No. 63/328,255, filed Apr. 7, 2022 (Atty Dkt No. 2021PF00898/44755.2271PV01), which is hereby incorporated by reference in its entirety as though fully set forth herein. Examples of pressure sensor housings and pressure sensor mounts (e.g., pressure sensor housings and/or pressure sensor mounts produced by additive manufacturing, 3D printing, or semiconductor fabrication techniques) can be found for example in U.S. Provisional Patent Application No. 63/330,380, filed Apr. 13, 2022 (Atty Dkt No. 2021PF00908/44755.2268PV01), U.S. patent application Ser. No. 17/188,012 to Burkett, filed Mar. 1, 2021, (Atty Dkt No. 2012P02343US03/44755.1227US03), U.S. Pat. No. 10,932,678 to Burkett, filed May 22, 2018, U.S. Pat. No. 9,974,446 to Burkett, and U.S. Provisional Patent Application No. 61/695,970 to Burkett, filed Aug. 31, 2012, each of which is hereby incorporated by reference in its entirety as though fully set forth herein. The pressure sensor may be fixed within the pressure sensor housing using adhesive, and mounted to the pressure sensor mount such that the sensing element (e.g., a diaphragm located near the distal end of sensor) is cantilevered, as described in U.S. Pat. No. 6,167,763, hereby incorporated by reference in its entirety as though fully set forth herein.

These descriptions are provided for exemplary purposes only and should not be considered to limit the scope of the metal ink conductor assembly. Certain features may be added, removed, or modified without departing from the spirit of the claimed subject matter.

For the purposes of promoting an understanding of the principles of the present disclosure, reference will now be made to the aspects illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one aspect may be combined with the features, components, and/or steps described with respect to other aspects of the present disclosure. Further, while the aspects of the present disclosure may be described with respect to a blood vessel, it will be understood that the devices, systems, and methods described herein may be configured for use in any suitable anatomical structure or body lumen including a blood vessel, blood vessel lumen, an esophagus, eustachian tube, urethra, fallopian tube, intestine, colon, and/or any other suitable anatomical structure or body lumen. In other aspects, the devices, systems, and methods described herein may be used to examine any number of anatomical locations and tissue types, including without limitation, organs including the liver, heart, kidneys, gall bladder, pancreas, lungs; ducts; intestines; nervous system structures including the brain, dural sac, spinal cord and peripheral nerves; the urinary tract; as well as valves within the blood vessels, chambers or other parts of the heart, and/or other systems of the body. In addition to natural structures, the device may be used to examine man-made structures such as, but without limitation, heart valves, stents, shunts, filters, and other devices. For the sake of brevity, however, the numerous iterations of these combinations will not be described separately.

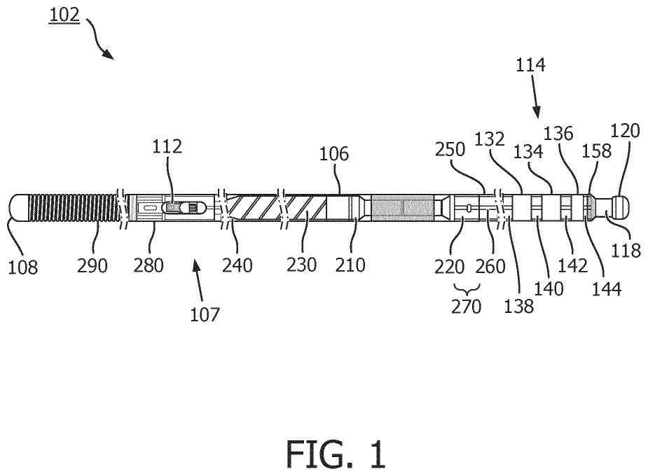

FIG. 1 is a diagrammatic top view of an intravascular device 102, according to aspects of the present disclosure. The intravascular device 102 may be an intravascular, intraluminal, or endoluminal device, such as a guidewire, a catheter, or a guide catheter sized and shaped for positioning within a blood vessel of a patient. The intravascular device 102 may include a sensor 112. For example, the sensor 112 may be a pressure sensor configured to measure a pressure of blood flow within the vessel of the patient. The intravascular device 102 includes the flexible elongate member 106. The sensor 112 is disposed at the distal portion 107, also referred to as a distal subassembly, of the flexible elongate member 106. The sensor 112 may be mounted at the distal portion 107 within a housing 280 in some aspects. A flexible tip coil 290 extends between the housing 280 and the distal end 108. The connection portion 114 is disposed at the proximal portion 109, also referred to as a proximal subassembly, of the flexible elongate member 106. The connection portion includes the conductive portions 132, 134, 136, spaced by non-conductive portions 138, 140, 142, and 144. In some aspects, the conductive portions 132, 134, 136 may be conductive ink that is printed and/or deposited around the flexible elongate member 106. In some aspects, the conductive portions 132, 134, 136 may be conductive, metallic rings that are positioned around the flexible elongate member. The locking section 118 and knob or retention section 120 are disposed at the proximal portion 109 of the flexible elongate member 106.

The intravascular device 102 in FIG. 1 includes a distal core 210 and a proximal core 220. The distal core 210 and the proximal core 220 are metallic components forming part of the body of the intravascular device 102. For example, the distal core 210 and the proximal core 220 are flexible metallic rods that provide structure for the flexible elongate member 106. The diameter of the distal core 210 and the proximal core 220 that electrically and mechanically couples the distal core 210 to the proximal core 220 may vary along its length. A joint between the distal core 210 and proximal core 220, which electrically and mechanically couples the distal core 210 to the proximal core 220, is surrounded and contained by a hypotube 215, which is a tubular member.

In some aspects, the intravascular device 102 includes a distal assembly and a proximal assembly that are electrically and mechanically joined together, which results in electrical communication between the sensor 112 and the conductive portions 132, 134, 136. For example, pressure data obtained by the sensor 112 (in this example, sensor 112 is a pressure sensor) may be transmitted to the conductive portions 132, 134, 136. Control signals from a computer in communication with the intravascular device 102 may be transmitted to the sensor 112 via the conductive portions 132, 134, 136. The distal subassembly may include the distal core 210. The distal subassembly may also include the sensor 112, conductive members 230, and/or one or more layers of insulative polymer/plastic 240 surrounding the conductive members 230 and the distal core 210. For example, the polymer/plastic layer(s) may protect the conductive members 230. The proximal subassembly may include the proximal core 220. The proximal subassembly may also include one or more layers of polymer layer(s) 250 (hereinafter polymer layer 250) surrounding the proximal core 220 and/or conductive ribbons 260 embedded within the one or more layers of polymer layer(s) 250. In some aspects, the proximal subassembly and the distal subassembly may be separately manufactured. During the assembly process for the intravascular device 102, the proximal subassembly and the distal subassembly may be electrically and mechanically joined together. As used herein, flexible elongate member may refer to one or more components along the entire length of the intravascular device 102, one or more components of the proximal subassembly (e.g., including the proximal core 220, etc.), and/or one or more components the distal subassembly (e.g., including the distal core 210, etc.).

In various aspects, the intravascular device 102 may include one, two, three, or more core wires, also referred to as core members, extending along its length. For example, a single core wire may extend substantially along the entire length of the flexible elongate member 106. In such aspects, the locking section 118 and the knob or retention section 120 may be integrally formed at the proximal portion of the single core wire. The sensor 112 may be secured at the distal portion of the single core wire. In other aspects, such as the illustration in FIG. 1, the locking section 118 and the knob or retention section 120 may be integrally formed at the proximal portion of the proximal core 220. The sensor 112 may be secured at the distal portion of the distal core 210. The intravascular device 102 includes one or more conductive members 230 in communication with the sensor 112. For example, the conductive members 230 may be one or more electrical wires that are directly in communication with the sensor 112. In some instances, the conductive members 230 are electrically and mechanically coupled to and in electrical communication with the sensor 112 by, e.g., soldering. In some instances, the conductive members 230 include two or three electrical wires (e.g., a bifilar cable or a trifilar cable). An individual electrical wire may include a bare metallic conductor surrounded by one or more insulating layers. The conductive members or filars 230 may extend along the length of the distal core 210. For example, at least a portion of the conductive members 230 may be spirally wrapped around the distal core 210.

The intravascular device 102 includes one or more conductive ribbons 260 at the proximal portion of the flexible elongate member 106. The conductive ribbons 260 are embedded within polymer layer(s) 250. The conductive ribbons 260 are directly in communication with the conductive portions 132, 134, and/or 136. In some instances, the conductive members 230 are electrically and mechanically coupled to and in electrical communication with the sensor 112 by, e.g., soldering. In some instances, the conductive portions 132, 134, and/or 136 include conductive ink (e.g., metallic nano-ink, such as silver or gold nano-ink) that is deposited or printed directed over the conductive ribbons 260.

As described herein, electrical communication between the conductive members 230 and the conductive ribbons 260 may be established at the connection region 270 of the flexible elongate member 106. By establishing electrical communication between the conductive members 230 and the conductive ribbons 260, the conductive portions 132, 134, 136 may be in electrically communication with the sensor 112.

In some aspects represented by FIG. 1, intravascular device 102 includes the locking section 118 and the knob or retention section 120. To form the locking section 118, a machining process may remove the polymer layer 250 and the conductive ribbons 260 in the locking section 118, and shape proximal core 220 in the locking section 118 to the desired shape. As shown in FIG. 1, the locking section 118 includes a reduced diameter while the knob or retention section 120 has a diameter substantially similar to that of proximal core 220 in the connection portion 114. In some instances, because the machining process removes conductive ribbons in locking section 118, proximal ends of the conductive ribbons 260 would be exposed to moisture and/or liquids, such as blood, saline solutions, disinfectants, and/or enzyme cleaner solutions, an insulation layer 158 is formed over the proximal end portion of the connection portion 114 to insulate the exposed conductive ribbons.

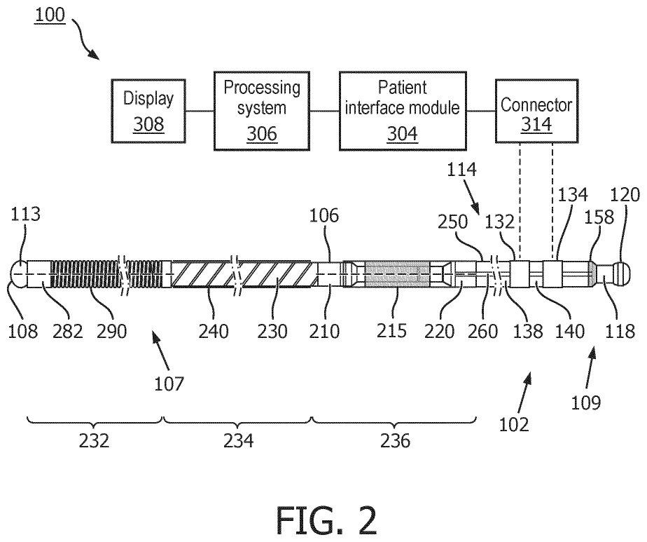

FIG. 2 is a diagrammatic side view of an intraluminal (e.g., intravascular) sensing system 100 that includes an intravascular device 102 includes conductive members 230 (e.g., a multi-filar electrical conductor bundle) and conductive ribbons 260, according to aspects of the present disclosure. The intravascular device 102 may be an intravascular guidewire sized and shaped for positioning within a blood vessel of a patient. The intravascular device 102 includes a distal end 108 and a sensor 113. For example, the sensor 113 may be a pressure sensor and/or flow sensor configured to measure a pressure of blood flow within the vessel of the patient, or another type of sensor including but not limited to a temperature or imaging sensor, or combination sensor measuring more than one property. For example, the flow data obtained by a flow sensor may be used to calculate physiological variables such as coronary flow reserve (CFR). The intravascular device 102 includes a flexible elongate member 106. The sensor 113 is disposed at a distal portion 107 of the flexible elongate member 106. The sensor 113 may be mounted at the distal portion 107 within a housing 282 in some aspects. A flexible tip coil 290 extends proximally from the housing 282 at the distal portion 107 of the flexible elongate member 106. A connection portion 114 located at a proximal end of the flexible elongate member 106 includes conductive portions 132, 134. In some aspects, the conductive portions 132, 134 may be conductive ink that is printed and/or deposited around the connection portion 114 of the flexible elongate member 106. In some aspects, the conductive portions 132, 134 are conductive, may be metallic bands or rings that are positioned around the flexible elongate member. A locking area is formed by a collar or locking section 118 and knob or retention section 120 are disposed at the proximal portion 109 of the flexible elongate member 106.

The intravascular device 102 in FIG. 2 includes core wire including a distal core 210 and a proximal core 220. In some instances, the distal core 210 and the proximal core 220 are metallic components forming part of the body of the intravascular device 102. For example, the distal core 210 and the proximal core 220 may be flexible metallic rods that provide structure for the flexible elongate member 106. The distal core 210 and/or the proximal core 220 may be made of a metal or metal alloy. For example, the distal core 210 and/or the proximal core 220 may be made of stainless steel, Nitinol, Titanium, nickel-cobalt-chromium-molybdenum alloy (e.g., MP35N), and/or other suitable materials. In some instances, the distal core 210 and/or the proximal core 220 may be made from a stiff graphite or similar composite material, such as carbon fiber, Kevlar, etc. In some aspects, the distal core 210 and the proximal core 220 are made of the same material. In other aspects, the distal core 210 and the proximal core 220 are made of different materials. The diameter of the distal core 210 and the proximal core 220 may vary along their respective lengths. A joint between the distal core 210 and proximal core 220 is surrounded and contained by a hypotube 215. The sensor 113 may in some cases be positioned at a distal end of the distal core 210.

In some aspects, the intravascular device 102 includes a distal subassembly and a proximal subassembly that are electrically and mechanically joined together, which creates an electrical communication between the sensor 113 and the conductive portions 132, 134. For example, flow data obtained by the sensor 113 (in this example, sensor 113 is a flow sensor) may be transmitted to the conductive portions 132, 134. In an exemplary aspect, the sensor 113 is a single ultrasound transducer element. The transducer element emits ultrasound signals and receives echoes. The transducer element generates electrical signals representative of the echoes. The signal carrying filars carry this electrical signal from the sensor at the distal portion to the connector at the proximal portion. The processing system 306 processes the electrical signals to extract the flow velocity of the fluid.

Control signals from the processing system 306 (e.g., a processor circuit of the processing system 306) in communication with the intravascular device 102 may be transmitted to the sensor 113 via a connector 314 that is attached to the conductive portions 132, 134. The distal subassembly may include the distal core 210. The distal subassembly may also include the sensor 113, the conductive members 230, and/or one or more layers of insulative polymer/plastic 240 surrounding the conductive members 230 and the distal core 210. For example, the polymer/plastic layer(s) may insulate and protect the conductive members of the conductive members 230. The proximal subassembly may include the proximal core 220. The proximal subassembly may also include one or more polymer layers 250 (hereinafter polymer layer 250) surrounding the proximal core 220 and/or conductive ribbons 260 embedded within the one or more insulative and/or polymer layer 250. In some aspects, the proximal subassembly and the distal subassembly are separately manufactured. During the assembly process for the intravascular device 102, the proximal subassembly and the distal subassembly may be electrically and mechanically joined together. As used herein, flexible elongate member may refer to one or more components along the entire length of the intravascular device 102, one or more components of the proximal subassembly (e.g., including the proximal core 220, etc.), and/or one or more components the distal subassembly (e.g., including the distal core 210, etc.). Accordingly, flexible elongate member may refer to the combined proximal and distal subassemblies described above. The joint between the proximal core 220 and distal core 210 is surrounded by the hypotube 215, which is a tubular member.

In various aspects, the intravascular device 102 may include one, two, three, or more core wires extending along its length. For example, a single core wire may extend substantially along the entire length of the flexible elongate member 106. In such aspects, the locking section 118 and the knob or retention section 120 may be integrally formed at the proximal portion of the single core wire. The sensor 113 may be secured at the distal portion of the single core wire. In other aspects, such as the illustration in FIG. 2, the locking section 118 and the knob or retention section 120 may be integrally formed at the proximal portion of the proximal core 220. The sensor 113 may be secured at the distal portion of the distal core 210. The intravascular device 102 includes one or more conductive members 230 (e.g., a multi-filar conductor bundle or cable) in communication with the sensor 113. For example, the conductive members or filars 230 may be one or more electrical wires that are directly in communication with the sensor 113. In some instances, the conductive members 230 are electrically and mechanically coupled to and in electrical communication with the sensor 113 by, e.g., soldering. In some instances, the conductive members 230 includes two or three electrical wires (e.g., a bifilar cable or a trifilar cable). An individual electrical wire may include a bare metallic conductor surrounded by one or more insulating layers. The conductive members 230 may extend along the length of the distal core 210. For example, at least a portion of the conductive members 230 may be spirally wrapped around the distal core 210, minimizing or eliminating whipping of the distal core within tortuous anatomy.

The intravascular device 102 may include one or more conductive ribbons 260 at the proximal portion of the flexible elongate member 106. The conductive ribbons 260 are embedded within polymer layer 250. The conductive ribbons 260 are directly in communication with the conductive portions 132 and/or 134. In some instances, conductive members 230 are electrically and mechanically coupled to and in electrical communication with the sensor 113 by, e.g., soldering. In some instances, the conductive portions 132 and/or 134 includes conductive ink (e.g., metallic nano-ink, such as copper, silver, gold, or aluminum nano-ink) that is deposited or printed directed over the conductive ribbons 260.

As described herein, electrical communication between the conductive members 230 and the conductive ribbons 260 may be established at the connection portion 114 of the flexible elongate member 106. By establishing electrical communication between the conductive members 230 and the conductive ribbons 260, the conductive portions 132, 134 may be in electrical communication with the sensor 113.

In some aspects represented by FIG. 1, the intravascular device 102 includes a locking section 118 and knob or retention section 120. To form locking section 118, a machining process is used to remove the polymer layer 250 and conductive ribbons 260 in locking section 118 and to shape proximal core 220 in locking section 118 to the desired shape. As shown in FIG. 1, locking section 118 includes a reduced diameter while knob or retention has a diameter substantially similar to that of proximal core 220 in the connection portion 114. In some instances, because the machining process removes conductive ribbons in locking section 118, proximal ends of the conductive ribbons 260 would be exposed to moisture and/or liquids, such as blood, saline solutions, disinfectants, and/or enzyme cleaner solutions, an insulation layer 158 is formed over the proximal end portion of the connection portion 114 to insulate the exposed conductive ribbons 260.

In some aspects, a connector 314 provides electrical connectivity between the conductive portions 132, 134 and a Patient Interface Monitor (PIM) 304. The PIM 304 may in some cases connect to a console or processing system 306, which includes or is in communication with a display 308. It is noted that the pressure-sensing guidewire of FIG. 1 can also be similarly in communication with the same or a different connector 314, PIM 304, processing system 306, and display 308.

The intraluminal sensing system 100 may be deployed in a catheterization laboratory having a control room. The processing system 306 may be located in the control room. Optionally, the processing system 306 may be located elsewhere, such as in the catheterization laboratory itself. The catheterization laboratory may include a sterile field while its associated control room may or may not be sterile depending on the procedure to be performed and/or on the health care facility. In some aspects, the intravascular device 102 may be controlled from a remote location such as the control room, such that an operator is not required to be in close proximity to the patient.

The intravascular device 102, PIM 304, and display 308 may be communicatively coupled directly or indirectly to the processing system 306. These elements may be communicatively coupled to the processing system 306 via a wired connection such as the conductive members 230, which is a standard copper multi-filar conductor bundle. The processing system 306 may be communicatively coupled to one or more data networks, e.g., a TCP/IP-based local area network (LAN). In other aspects, different protocols may be utilized such as Synchronous Optical Networking (SONET). In some cases, the processing system 306 may be communicatively coupled to a wide area network (WAN).

The PIM 304 transfers the received signals to the processing system 306 where the information is processed and displayed (e.g., as physiology data in graphical, symbolic, or alphanumeric form) on the display 308. The console or processing system 306 may include a processor and a memory. The processing system 306 may be operable to facilitate the features of the intraluminal sensing system 100 described herein. For example, the processor may execute computer readable instructions stored on the non-transitory tangible computer readable medium.

The PIM 304 facilitates communication of signals between the processing system 306 and the intravascular device 102. The PIM 304 may be communicatively positioned between the processing system 306 and the intravascular device 102. In some aspects, the PIM 304 performs preliminary processing of data prior to relaying the data to the processing system 306. In examples of such aspects, the PIM 304 performs amplification, filtering, and/or aggregating of the data. In an aspect, the PIM 304 also supplies high-and low-voltage DC power to support operation of the intravascular device 102 via the conductive members 230.

A multi-filar cable or transmission line bundle, such as conductive members 230, may include a plurality of conductors, including one, two, three, four, five, six, seven, or more conductors. In the example shown in FIG. 2, the conductive members 230 includes two straight portions 232 and 236, where the conductive members 230 lies parallel to a longitudinal axis of the flexible elongate member 106, and a spiral portion 234, where the conductive members 230 is wrapped around the exterior of the flexible elongate member 106 and then overcoated with the insulative polymer/plastic 240. Communication, if any, along the conductive members 230 may be through numerous methods or protocols, including serial, parallel, and otherwise, where one or more filars of the conductive members 230 carry signals. One or more filars of the conductive members 230 may also carry direct current (DC) power, alternating current (AC) power, or serve as a ground connection.

The display or monitor 308 may be a display device such as a computer monitor or other type of screen. The display or monitor 308 may be used to display selectable prompts, instructions, and visualizations of imaging data to a user. In some aspects, the display 308 may be used to provide a procedure-specific workflow to a user to complete an intraluminal imaging procedure.

Although FIGS. 1 and 2 show the details of particular intraluminal measurement devices, it should be understood that these examples are illustrative rather than limiting, and the present disclosure can apply to other types of intravascular devices than those shown or described herein.

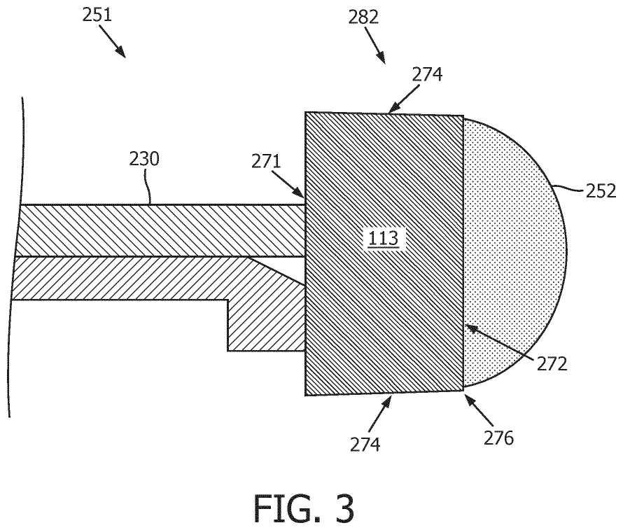

FIG. 3 is a diagrammatic cross-sectional view of an example sensor assembly 251, which may for example be included in the intravascular device 102 of FIG. 2, according to aspects of the present disclosure. More specifically, FIG. 3 illustrates a sensor assembly 251 that includes a sensing component 113, a housing 282, and an acoustic matching layer 252. As indicated by the positions of the sensing component 113 and the housing 282 illustrated in FIG. 2, the sensor assembly 251 may be included in a distal portion of the intravascular device 102 such that the surface 272 of the sensing component 113 faces distally.

As illustrated in FIG. 3, the sensing component 113 is positioned within the housing 280 and includes a proximal surface 271, an opposite, distal surface 272, and a side surface 274. In some embodiments, one or more of the proximal surface 271, the distal surface 272, or the side surface 274 may be coated in an insulating layer 276. The insulating layer 276 may for example be formed from parylene, which may be deposited on one or more surfaces. The insulating layer 276 may additionally or alternatively be formed from any other suitable insulating material. In some embodiments, the insulating layer 276 may prevent a short (e.g., an electrical failure), which may otherwise be caused by contact between a conductive portion of the sensing component 113 and the housing 282, which may be formed with a metal. As used herein, references to the distal surface 272 encompass the insulating layer 276 in embodiments where a distal end of the sensing component 113 is covered by the insulating layer 276, references to the proximal surface 271 encompass the insulating layer in embodiments where a proximal end of the sensing component 113 is covered by the insulating layer 276, and references to the side surface 274 encompass the insulating layer in embodiments where the side of the sensing component 113 is covered by the insulating layer 276 unless indicated otherwise.

In some embodiments, the sensing component 113 may include a transducer element, such as an ultrasound transducer element on the distal surface 272 such that the transducer element faces distally and may be used by the sensing component 113 to obtain sensor data corresponding to a structure distal of the sensing component 113. The sensing component 113 may additionally or alternatively include a transducer element on the proximal surface 271 such that the transducer faces proximally and may be used to obtain sensor data corresponding to a structure proximal of the sensing component. A transducer element may additionally or alternatively be positioned on a side surface 274 (e.g., on a perimeter or circumference) of the sensing component 113 in some embodiments. In some embodiments, a transducer and its associated electrodes and electrical connection points may form the entire sensing component 113, such that all surfaces of the sensing component 113 comprise the transducer.

As further illustrated, the sensing component 113 is coupled to the multi-filar conductor bundle 230, and at least a portion (e.g., a distal portion) of the multi-filar conductor bundle 230 extends through the housing 282. In some embodiments, the multi-filar conductor bundle 230 and the sensing component 113 may be physically (e.g., mechanically) coupled. Further, one or more filars (e.g., conductive members) of the multi-filar conductor bundle 230 may electrically couple to (e.g., be in electrical communication) with the sensing component 113. In particular, one or more filars of the multi-filar conductor bundle 230 may couple to an element, such as a transducer (e.g., an ultrasound transducer), of the sensing component 113 and may provide power, control signals, an electrical ground or signal return, and/or the like to the element. As described above, such an element may be positioned on the distal surface 272 of the sensor. In that regard, in some embodiments, one or more filars of the multi-filar conductor bundle 230 may extend through a cutout or hole in the sensing component 113 (e.g., in at least the proximal surface 271) to establish electrical communication with an element on the distal surface 272 of the sensor. Filars may additionally or alternatively wrap around the side surface 274 to establish electrical communication with the element on the distal surface 272. Moreover, in some embodiments, filars of the multi-filar conductor bundle 230 may terminate at and/or electrically couple to the proximal surface 271 (e.g., to an element on the proximal surface 271) of the sensing component 113. Further, in some embodiments, a subset of the filars of the multi-filar conductor bundle 230 may extend to the distal surface 272 and/or electrically couple to an element at the distal surface 272, while a different subset of the filars may electrically couple to an element at the proximal surface 271, for example.

In some embodiments, the multi-filar conductor bundle 230 may be coated in the insulating layer 276. In some embodiments, for example, the multi-filar conductor bundle 230 and the sensing component 113 may be coupled together in a sub-assembly before being positioned in the housing 282. In such embodiments, the insulating layer 276 may be applied (e.g., coated and/or deposited) onto the entire sub-assembly, resulting in an insulating layer 276 on both the sensing component 113 and the multi-filar conductor bundle 230.

In some embodiments, the acoustic matching layer 252 may be positioned on (e.g., over) the distal surface 272 of the sensing component 113. In particular, the acoustic matching layer 252 may be disposed directly on the sensing component 113, or the acoustic matching layer 252 may be disposed on the insulating layer 276 coating the sensing component 113. Further, the acoustic matching layer 252 may be disposed on a transducer element (e.g., an ultrasound transducer element) positioned on the sensing component (e.g., the distal surface 272) and/or at least a portion of a conductive filar of the multi-filar conductor bundle 230 that is in communication with the transducer element, such as a filar extending through a hole or along a side of the sensing component 113. To that end, the acoustic matching layer 252 may contact and/or at least partially surround the portion of the conductive filar and/or the transducer element. Moreover, the acoustic matching layer 252 may provide acoustic matching to the sensing component 113 (e.g., to an ultrasound transducer of the sensing component 113). For instance, the acoustic matching layer 252 may minimize acoustic impedance mismatch between the ultrasound transducer and a sensed medium, such as a fluid and/or a lumen that the intravascular device 102 is positioned within. In that regard, the acoustic matching layer 252 may be formed from any suitable material, such as a polymer or an adhesive, to provide acoustic matching with the sensing component 113. The portion of the acoustic matching layer 252 positioned on the distal surface 272 may include and/or be formed from the same material as a portion of the acoustic matching layer positioned on the side surface 274 and/or the proximal surface 271. Further, the acoustic matching layer 252 may be applied to the sensing component 113 before or after the sensing component 113 is positioned within the housing 282 during assembly of the sensor assembly 251. In this regard, the portion of the acoustic matching layer 252 positioned on the distal surface 272 and the portion of the acoustic matching layer positioned on the side surface 274 and/or the proximal surface 271 may be included in the sensor assembly 251 in the same or different steps. Further, in addition to the one or more materials the acoustic matching layer 252 is formed from, the acoustic matching layer 252 may provide acoustic matching with the sensing component 113 via one or more dimensions of the acoustic matching layer 252.

In some embodiments, the sensor assembly 251 may include an atraumatic tip, such as the distal tip 108 illustrated in FIG. 1. In some embodiments, the distal tip 108 may include the same material as the acoustic matching layer 252. In some embodiments, the distal tip may include a different material than the acoustic matching layer 252. Additionally or alternatively the distal tip 108 may be formed from one or more layers of materials. The layers may include different materials and/or different configurations (e.g., shape and/or profile, thickness, and/or the like). Further, the distal tip 108 may be arranged to cover the distal surface 272 of the sensing component 113. In some embodiments, the distal tip 108 may also cover a distal end 272 of the housing 282. Moreover, while the distal tip 108 is illustrated as having a domed shape, embodiments are not limited thereto. In this regard, the distal tip 108 may include a flattened profile or any suitable shape. In some embodiments, the entire sensing component 113 may be positioned within (e.g., surrounded by the continuous surface of) the housing 282.

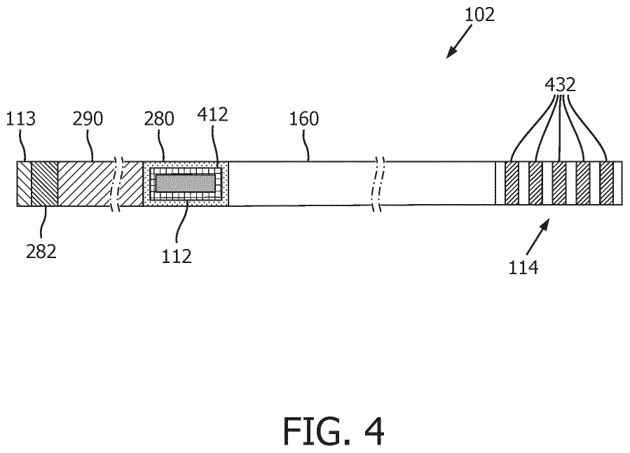

FIG. 4 is a diagrammatic cross-sectional view of an intraluminal (e.g., intravascular) sensing device 102 that includes both a pressure sensor 112 and a flow sensor 113, according to aspects of the present disclosure. In the example shown in FIG. 4, the intraluminal sensing device includes a flow sensor 113 positioned at a distal end of the flexible elongate member 106. The flow sensor 113 may be at least partially contained within a flow sensor housing 282. A coil 290 is disposed proximal of the flow sensor housing 282. Proximal of the coil is a pressure sensor housing 280, which may at least partially enclose a pressure sensor mount 412, to which a pressure sensor 112 is attached. At a proximal end of the flexible elongate member 106 is a connection portion 114 comprising a plurality of conductive portions 432, such as conductive bands as described above. In an example, a pressure sensor 112 may be operated by three conductor paths, such as three conductive wires or filars, or two conductive wires or filars plus a conductive core wire. Thus, three of the conductive bands 432 may be electrically connected to the pressure sensor 112. It is noted that the conductive wires or filars could have circular cross section or a flatted ribbon-like cross section, or other cross section, and could comprise multiple segments of different cross section and/or different materials.

In an example, a flow sensor 113 may be operated by two conductor paths, such as two conductive wires or filars, or one conductive wire or filar plus a conductive core wire. Thus, two of the conductive bands 432 may be electrically connected to the flow sensor 113. Thus, the connection portion 114 may include five conductive bands 432. In some instances, there could be four conductive bands 432. 2 for the pressure sensor and 2 for the flow sensor. The pressure sensor could still be connected to 3 wires or filars, but one of the wires or filars would be grounded (e.g., to the core wire) and thus not need a conductive band. However, in some embodiments, the two conductive paths that operate the flow sensor 113 may be shared by the pressure sensor (e.g., by operating the pressure sensor at some times and operating the flow sensor at other times). In such embodiments, there may be three conductive bands 432 in the connection portion 114. In some instances, there could be two conductive bands 432 (both shared by the pressure sensor and the flow sensor). The pressure sensor could still be connected to 3 wires or filars, but one of the wires or filars could be grounded (e.g., to the core wire) and thus not need a conductive band. Depending on the implementation, other numbers of conductive bands or conductive paths may be used instead or in addition.

Other numbers or arrangements of sensors may also be used. In an intraluminal sensing device that includes a first sensor and a second sensor, the first sensor can include any suitable sensing modality (e.g., pressure, flow, temperature, imaging, etc.). The second sensor can also be any suitable modality (e.g., pressure, flow, temperature, imaging, etc.), whether the same or different than the modality of the first sensor.

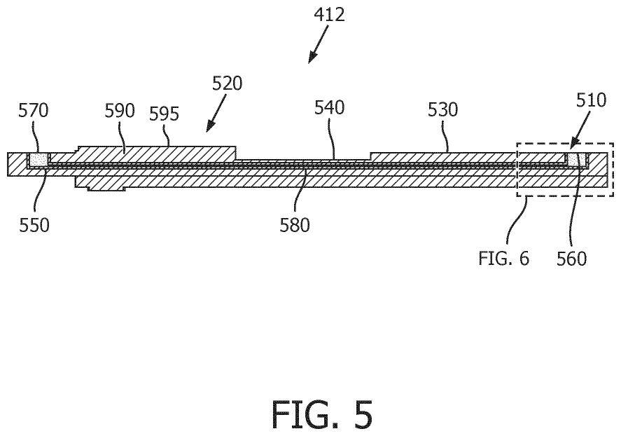

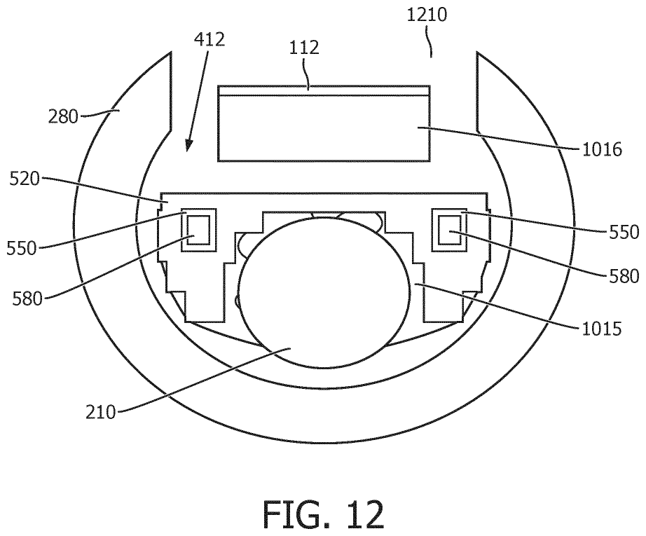

FIG. 5 is a diagrammatic cross-sectional side view of a sensor mount 412 (e.g., a pressure sensor mount) with embedded signal-carrying conductive material 510, according to aspects of the present disclosure. In an example, the sensor mount 412 includes a sensor mount body 520, which includes a sensor mounting platform 530 and a sensor cantilever recess 540. The sensor mount body 520 may for example be made of a metallic material. In other embodiments, the sensor mount body 520 may be made of an electrically insulating material, or a combination of electrically insulating and conductive (e.g., metallic) materials. As shown below in FIG. 12, the sensor mount 412 may be attached to the core wire (e.g., distal core wire 210 of FIG. 1), within a sensor housing (e.g., sensor housing 280 of FIG. 1).

The signal-carrying conductive material 510 may be embedded within the sensor mount body 520, and may be surrounded by an insulating material 550 which may be in contact with the material of the sensor mount body 520 and also in contact with the signal-carrying conductive material 510 to, for example, prevent electrical contact between the sensor mount body 520 and the signal-carrying conductive material 510. In the example shown in FIG. 5, the signal-carrying conductive material 510 includes one or more proximal terminals 560, one or more distal terminals 570, one or more embedded conductors 580 that electrically connect the proximal terminals 560 to the distal terminals 570. The proximal terminals 560 and distal terminals 570 thus form part of the outer surface of the sensor mount body 520, such that electrical signals can be routed from the proximal end to the distal end of the sensor mount 412, without the need to run insulated wires or filars alongside the sensor mount 412 within the sensor housing 280. This may for example allow the sensor housing 280 to have smaller dimensions that fit more closely against the sensor mount 412, or may allow the sensor mount 412 and sensor housing 280 to be fabricated together as a single object.

Thus, for some implementations, from the inside out, FIG. 5 shows one or more channels of signal-carrying conductive material 510, surrounded by electrically insulating (e.g., dielectric) material 550, then which is then surrounded by conductive material 590 (which may be the same or a different material than the signal-carrying conductive material 510), forming the outer surface 595 of the sensor mount body 520 and thus the outer surface of the sensor mount 412. Depending on the implementation, the conductive material 590 may or may not be signal-carrying. In cases where it is signal-carrying, the signal carried by the conductive material 590 may be different than any of the signals carried by the signal-carrying conductive material 510.

Depending on the implementation, the signal carrying conductive material 510 may make up the structure of the sensor mount 412, or the signal carrying conductive material 510 may form part of the structure of the sensor mount 412, or the signal carrying conductive material 510 may be positioned inside/within an outer perimeter of the sensor mount 412, as shown in FIGS. 5 and 12. In some instances, the structure of the sensor mount 412 is homogenous and used to transmit electrical signals. Still other arrangements may be used, including combinations of conductive and insulating materials (e.g., in alternating layers or other arrangements) that serve the same or a similar function of transporting signals to and from a distal component through the sensor mount 412.

Thus, for a combination intraluminal physiology sensing device incorporating two or more sensors, the sensor mount 412 includes conductive pathways that carries signals for another one of the sensors (e.g., a sensor located distal of and/or physically spaced from the sensor mount 412).

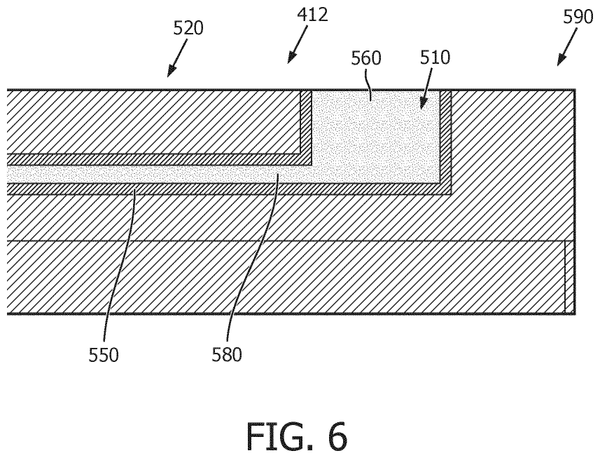

A detail region, marked by a dotted rectangle, is shown below in FIG. 6 at a larger magnification.

One or a plurality of sensors can be mounted on the sensor mount (e.g., sensor mount 412) so that the sensor mount physically supports the one or plurality of sensors. In some instances, the housing 282 can also be a sensor mount. A first sensor (e.g., the pressure sensor 112 of FIG. 4) is spaced from a second sensor (e.g., the flow sensor 113 of FIG. 4). In some instances, the first sensor is mounted on the sensor mount and the second sensor is not mounted on the sensor mount (e.g., the second sensor is spaced from the sensor mount). In some instances, the second sensor is mounted on the sensor mount and the first sensor is not mounted on the sensor mount (e.g., the first sensor is spaced from the sensor mount 412). In some instances, the first sensor, the second sensor, and/or other sensors are mounted on the sensor mount. In some instances, the sensor mount can include embedded conductive pathways (e.g., forming part of the structure of the sensor mount) that carries signals for the second sensor. In some instances, the sensor mount can include embedded conductive pathways that carries signals for the first sensor. In some instances, the sensor mount can include embedded conductive pathways that carries signals for the first sensor, the second sensor, and/or other sensors.

FIG. 6 is a diagrammatic cross-sectional view of the detail region of a sensor mount 412 (e.g., a pressure sensor mount) with embedded signal-carrying conductive material 510, according to aspects of the present disclosure. Visible are the sensor mount body 520, signal-carrying conductive material 510, proximal terminal 560, embedded conductor 580, and insulating material 550. In some embodiments, the sensor mount body 520 is made at least partially from the insulating material 550.

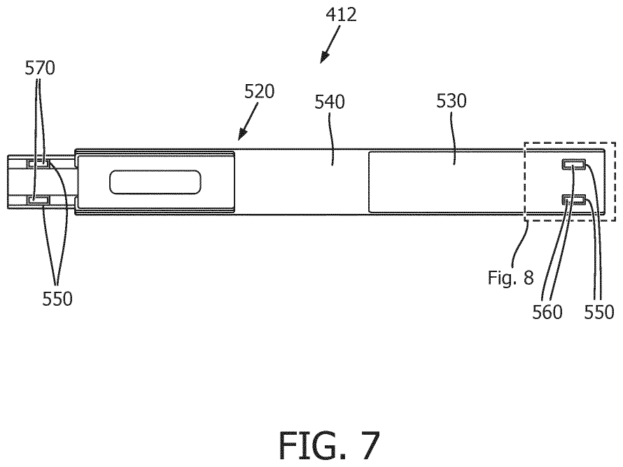

FIG. 7 is a diagrammatic top view of a sensor mount 412 (e.g., a pressure sensor mount) with embedded signal-carrying conductive material, according to aspects of the present disclosure. Visible are the sensor mount body 520, sensor mount platform 530, sensor cantilever recess 540, two proximal terminals 560, each surrounded by insulating material 550, and two distal terminals 570, each surrounded by insulating material 550. Depending on the implementation, other numbers of terminals (and their associated embedded conductors) may be provided instead or in addition to those shown in FIG. 7.

As illustrated, the terminals 560 and 570 are proximal and distal terminals, respectively. In other instances, the terminals can be anywhere along the length of the sensor mount 412. As illustrated, the terminals are exposed at the top surface of the sensor mount 412. In other instances, one or a plurality of the terminals can be exposed on any surface of the sensor mount 412, including the proximal surface, distal surface, right side surface, left side surface, top surface, bottom surface, or combinations thereof.

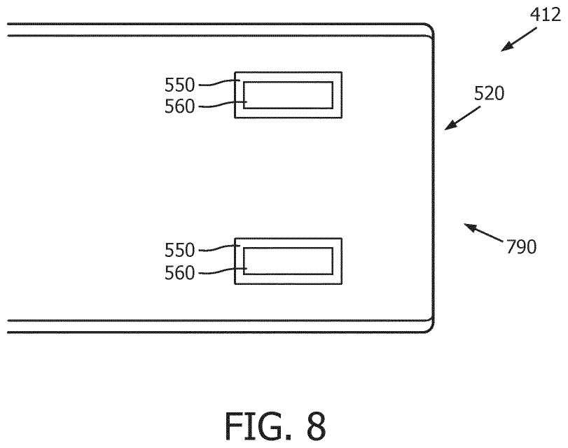

A detail region, marked by a dotted rectangle, is shown below in FIG. 8 at a larger magnification.

FIG. 8 is a diagrammatic cross-sectional view of the detail region 790 of an example sensor mount 412, according to aspects of the present disclosure. Visible are the sensor mount body 520 and two proximal terminals 560, each surrounded by insulating material 550. Depending on the implementation, other numbers of terminals (and their associated embedded conductors) may be provided instead or in addition to those shown in FIG. 8.

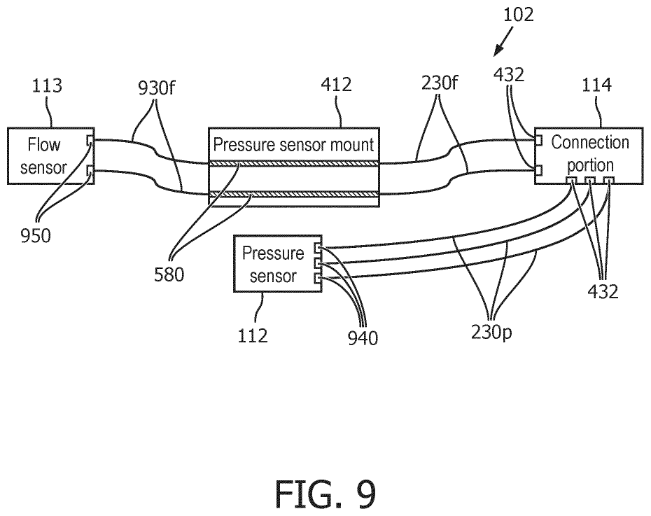

FIG. 9 is a schematic view of the wiring of an intraluminal (e.g., intravascular) sensing device 102 that includes both a pressure sensor 112 and a flow sensor 113, according to aspects of the present disclosure. The connection portion 114 of the intraluminal device 102 includes a number of conductive portions 432 (e.g., conductive bands), to which wires, filars, or conductors 230f and 230p are electrically connected. Three of the wires or filars 230p extending from the connection portion 114 connect to electrical contacts 940 on the pressure sensor 112, in order to carry power and signals between the connection portion 114 and the pressure sensor 112. Two of the wires or filars 230f extend from the connection portion 114 connect to the proximal ends of the embedded conductors 580 within the sensor mount 412 (e.g., by connecting to the proximal terminals 560 of FIG. 7). Two distal wires, filars, or conductors 930f are connected from the embedded conductors 580 of the sensor mount 412 (e.g., via the distal terminals 570 of FIG. 7) to the electrical contacts 950 of the flow sensor 113. In an example, the wires or filars 230p, 230f, and 930f are electrically bonded (e.g., by solder, conductive adhesive, ultrasonic welding, or other means) to the electrical contacts 940 and 950 or other terminals or contacts. It is understood that pressure sensor 112 may be positioned on and coupled to the pressure sensor mount 412.

Thus, electrical connectivity (e.g., for carrying power and signals) can be provided between the connection portion 114 and the flow sensor 113, without the need for wires or filars 230f to extend directly from the connection portion 114 to the flow sensor 113 by running alongside the pressure sensor mount 412. This arrangement may reduce both costs and manufacturing errors by allowing for simplified assembly procedures, while also allowing for a smaller gap between the pressure sensor mount 412 and a pressure sensor housing (e.g., sensor housing 280 of FIG. 4), since the gap does not need to accommodate the wires or filars. The resulting device may also be more robust during handling and use.

One or a plurality of five wires, filars, or conductors 230p, 230f shown in FIG. 9 could be a continuous length or multiple wires/conductors that are electrically and mechanical coupled. For example, part of the length could be a filar e.g., extending along/wrapped around distal core wire and part of the length could be, for example, a ribbon conductor extending along/embedded in polymer around the proximal core wire. A wire or filar, or a portion thereof, could be a bare metal conductor that is surrounded by polymer insulation (e.g., at the proximal portion of the guidewire as shown in FIGS. 1 and 2, or in other locations depending on the implementation). Exposed portions of the bare conductors at the ends (e.g., portions without insulation) can make electrical contact.

In the example shown in FIG. 9, the embedded conductors 580 are shown as rectangular, while other wires, filars, or conductors 230p, 230f, 930f are shown as flexible lines. For example, a filar could be wrapped around distal core wire or outed around or adjacent to other components, whereas the embedded conductors 580 are fixed within the pressure sensor mount 412. The power and/or signals carried by the distal wires or filars 930f and/or embedded conductors 580 may include, but are not limited to, power from the PIM 304 and/or processing system 306 (see FIG. 2) to the sensor, command or controls signals from PIM 304 and/or processing system 306 to the sensor, or obtained data from the sensor being transmitted back to the PIM 304 and/or processing system 306, or electrical grounds, for any suitable sensing modality, including but not limited to pressure, flow, temperature, imaging, etc. Sensors 112 and 113 can be different from one another (e.g., structurally/physically distinct sensors), can be facing different directions (e.g., one forward facing and one side facing), can be different sensing modalities, etc. For example, sensor 112 may be a pressure sensor and sensor 13 may be a flow sensor. In other aspects, sensors 112 and 113 may be similar or identical to one another.

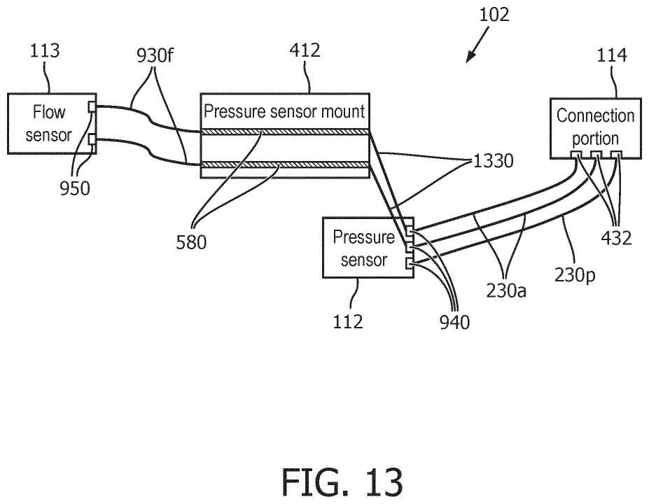

Other numbers or routings of wires, filars, embedded conductors, or other conductive pathways may be provided instead of or in addition to those shown in FIG. 13, without departing from the spirit of the present disclosure. In some aspects, the pressure sensor 112 may not be positioned on the pressure sensor mount 412. Thus, in a combination intraluminal physiology sensing device, the sensor mount for a first of the sensors includes conductive materials that carry signals for a different, second sensor of the device (e.g., a sensor that is not supported by the sensor mount and/or that is spaced from the sensor mount and/or the first sensor).

The flow signal pathways each include a combination of conductive segments 930f, 580, 230f or other segments that extend between the flow sensor 113 and the connection portion 114 to provide a continuous pathway for electrical signals associated with the flow sensor 113 (e.g., from the flow sensor 113 to the PIM 304 or processing system 306 of FIG. 2, from the PIM 304 or processing system 306 to the flow sensor 113, and/or from the flow sensor 113 to an electrical ground). Some of the conductive segments may be filars, wires, wire ribbons, conductive inks, conductive bands, or otherwise. Importantly, one of the conductive segments in each flow signal pathway is the signal-carrying conductive material embedded within the sensor mount. There can be one or multiple flow signal pathways (e.g., the two flow signal pathways illustrated in FIG. 9).

The pressure signal pathways each include a combination of conductive segments 230p that extend between the pressure sensor and the connection portion to provide a continuous pathway for electrical signals (e.g., from the pressure sensor 112 to the PIM 304 or processing system 306, from the PIM 304 or processing system 306 to the pressure sensor 112, or from the pressure sensor 112 to an electrical ground). Some of the conductive segments may be filars, wires, wire ribbons, conductive inks, conductive bands, conductive core wires, or otherwise. There can be one or multiple pressure signal pathways (e.g., three pathways illustrated in FIG. 9, though one of those three could be grounded before reaching the connection portion 114).

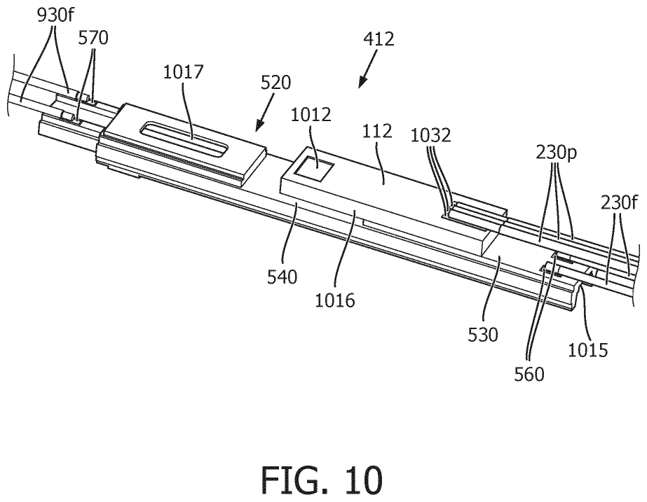

FIG. 10 is a diagrammatic perspective view of a sensor mount 412 (e.g., a pressure sensor mount) whose schematic wiring diagram is shown in FIG. 9, according to aspects of the present disclosure. The sensor mount 412 includes a sensor mount body 520, which includes a sensor mount platform 530 and sensor cantilever recess 540. A sensor 112 is attached to an outer surface of the sensor mount platform 530 (e.g., with an adhesive). In the example shown in FIG. 10, as in FIG. 9, three of the wires or filars 230p are connected to weld pads 1032 on the sensor 112, and two of the wires or filars 230f are connected to the proximal terminals 560 of the sensor mount 412. Two distal wires or filars 930f are connected to the distal terminals 570 of the sensor mount 412, to provide electrical connectivity to the flow sensor, located distal of the pressure sensor mount 412. It is noted that, proximal of the sensor mount 412, the wires or filars 230p and 230f are electrically coupled to, and form electrical pathways with, the connection portion 114, as shown above in FIG. 9. Similarly, distal of the sensor mount 412, the distal wires or filars 930f are electrically connected to, and for electrical pathways with, the flow sensor 113, as shown above in FIG. 9.

The sensor mount 412 also includes a core wire lumen 1015 (which may also be described as a core wire recess, core wire region, core wire space, or core wire opening), and an optional solder or glue hole 1017 to facilitate attachment of the sensor mount 412 to the core wire and/or to a shaping ribbon that is coupled to the sensor mount 412 and extends distal of the sensor mount. The core wire lumen 1015 may for example be configured to receive the distal core wire 210 (see FIG. 1). The weld pads 1032 of the sensor 112 are located on a fixed portion 214 of the sensor 112, a diaphragm or other sensing element 1012 is located on a cantilevered portion 1016 of the sensor 112, which is spaced from and disposed above the sensor cantilever recess 540. In some embodiments, the bottom surface of the sensor cantilever recess 540 may be deleted altogether, such that the bottom of the sensor cantilever recess 540 opens into the core wire lumen 1015.

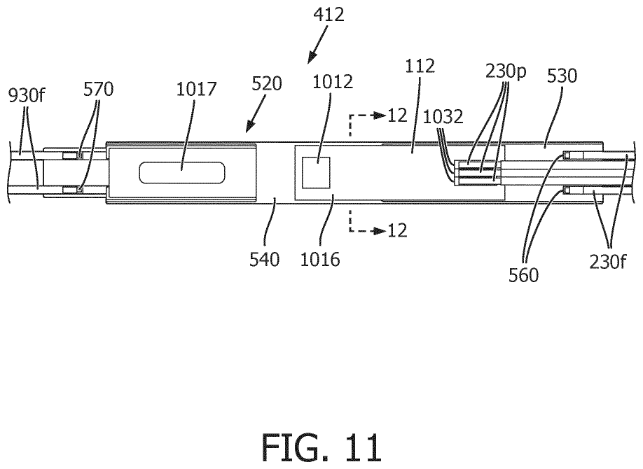

FIG. 11 is a diagrammatic top view of the sensor mount 412 of FIG. 10, according to aspects of the present disclosure. Visible are the sensor mount body 520, sensor mount platform 530, sensor cantilever recess 540, sensor 112, optional solder or glue hole 1017, weld pads 1032 of the sensor 112, fixed portion 214 of the sensor 112, and the sensing element 1012 on the cantilevered portion 1016 of the sensor 112, which is disposed above the sensor cantilever recess 540.

In the example shown in FIG. 11, as in FIGS. 9 and 10, three of the wires or filars 230p are connected to weld pads 1032 on the sensor 112, and two of the wires or filars 230f are connected to the proximal terminals 560 of the sensor mount 412. Two distal filars 930f are connected to the distal terminals 570 of the sensor mount 412, to provide electrical connectivity to the flow sensor, located distal of the pressure sensor mount 412.

A cross section line 12-12 shows the location of the cross-sectional view of FIG. 12.

FIG. 12 is a diagrammatic, lateral cross-sectional view of the sensor mount 412 of FIG. 11, taken along cross-section line 12 -12 (perpendicular to the longitudinal axis of the core wire), according to aspects of the present disclosure. Although FIGS. 2-8 and 10-11 show arrangement of components longitudinally, the lateral cross-section of FIG. 12 shows the arrangement of components laterally or radially, which is perpendicular to the longitudinal view.

Visible are the pressure sensor housing 280, which forms the outermost surface of the intraluminal device at this location. Radially inward from the housing 280, is the cantilever portion 1016 of the pressure sensor 112, which are disposed above the sensor mount body 520 and which are partially enclosed by the sensor housing 280, which includes an upper opening 1210 above the pressure sensor 112. The sensor mount body is positioned radially inward from, and is partially enclosed by, the pressure sensor housing 280. Within the sensor mount body 520 are two embedded conductors 580, each surrounded by a layer of insulating material 550. On the lower portion of the sensor mount body 520 is the core wire lumen 1015 (also describable as a trench, recess, depression, etc.), which partially encloses the distal core wire 210 at a lowermost portion of the sensor mount body 520.

This configuration does not require wires or filars passing through the sensor housing 280 adjacent to the sensor mount 412, and may thus allow for a smaller sensor housing 280, a larger sensor mount 412, and/or a closer fit between the sensor housing 280 and the sensor mount 412, or may allow the sensor mount 412 and sensor housing 280 to be combined into a single object.

In some instances, the housing and the mount are distinct components (e.g., that are coupled to one another). In some instances, the housing and the mount are the same component (as shown for example in U.S. Provisional Patent Application No. 63/330,380, filed Apr. 13, 2022 (Atty Dkt No. 2021PF00908/44755.2268PV01), incorporated by reference above). That is, the same component defines the outermost surface of the intraluminal device, as well as space for the core wire and the space for the pressure sensor. The signal-carrying conductive material and the electrically insulating (e.g., dielectric) material can be provided within such a same component.

FIG. 13 is a schematic view of the wiring of an intraluminal (e.g., intravascular) sensing device 102 that includes both a pressure sensor 112 and a flow sensor 113, according to aspects of the present disclosure. The connection portion 114 of the intraluminal device 102 includes a number of conductive portions 432 (e.g., conductive bands), to which wires, filars, or conductors 230a and 230p are electrically connected. Three wires or filars 230p and 230a, extend from the connection portion 114 connect to electrical contacts 940 on the pressure sensor 112, in order to carry power and signals between the connection portion 114 and the pressure sensor 112. However, unlike the embodiment shown in FIG. 9, no filars extend from the connection portion 114 connect to the proximal ends of the embedded conductors 580 within the sensor mount 412. Rather, two wire bonds or jumpers 1330 connect two of the electrical contacts 940 of the pressure sensor 112 to the proximal ends of the embedded conductors 580 (e.g., by connecting to the proximal terminals 560 of FIG. 7). Two distal wires, filars, or conductors 930f are connected from the embedded conductors 580 of the sensor mount 412 (e.g., via the distal terminals 570 of FIG. 7) to the electrical contacts 950 of the flow sensor 113. It is understood that pressure sensor 112 will, in most cases, be positioned on and coupled to the pressure sensor mount 412.

Thus, electrical connectivity (e.g., for carrying power and signals) can be provided between the connection portion 114 and the flow sensor 113, without the need for wires or filars to extend directly from the connection portion 114 to the flow sensor 113 by running alongside the pressure sensor mount 412. Furthermore, the two electrical pathways defined by wires or filars 230a are shared between the pressure sensor 112 and the flow sensor 113. This may be accomplished for example by operating the pressure sensor 112 and the flow sensor 113 at different times, at different frequencies, with different digital sequences, or otherwise.

A wire, filar, or conductor, or a portion thereof, could be a bare metal conductor that is surrounded by polymer insulation. The electrical pathways shown may comprise multiple conductive elements comprising the same, similar, or different materials. Other numbers of wires or filars 230p, 230a, or 950f, embedded conductors 580, jumpers 1330, or other conductive pathways may be provided instead of or in addition to those shown in FIG. 13.

The flow signal pathways each include a combination of conductive segments 930f, 580, 1330, 230a or other segments that extend between the flow sensor 113 and the connection portion 114 to provide a continuous pathway for electrical signals associated with the flow sensor 113 (e.g., from the flow sensor 113 to the PIM 304 or processing system 306 of FIG. 2, from the PIM 304 or processing system 306 to the flow sensor 113, and/or from the flow sensor 113 to an electrical ground). Some of the conductive segments may be filars, wires, wire ribbons, conductive inks, conductive bands, or otherwise. Importantly, one of the conductive segments in each flow signal pathway is the signal-carrying conductive material embedded within the sensor mount. There can be one or multiple flow signal pathways (e.g., the two flow signal pathways illustrated in FIG. 13).

The pressure signal pathways each include a combination of conductive segments 230a, 230p that extend between the pressure sensor and the connection portion to provide a continuous pathway for electrical signals (e.g., from the pressure sensor 112 to the PIM 304 or processing system 306, from the PIM 304 or processing system 306 to the pressure sensor 112, or from the pressure sensor 112 to an electrical ground). Some of the conductive segments may be filars, wires, wire ribbons, conductive inks, conductive bands, conductive core wires, or otherwise. There can be one or multiple pressure signal pathways (e.g., three pathways illustrated in FIG. 13, though one of those three could be grounded before reaching the connection portion 114).

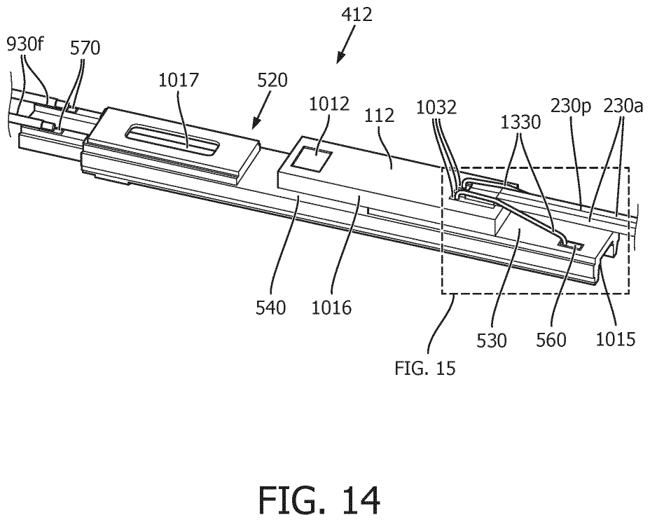

FIG. 14 is a diagrammatic perspective view of a sensor mount 412 (e.g., a pressure sensor mount) whose schematic wiring diagram is shown in FIG. 13, according to aspects of the present disclosure. The sensor mount 412 includes a sensor mount body 520, which includes a sensor mount platform 530 and sensor cantilever recess 540. A sensor 112 is attached to the sensor mount platform 530. In the example shown in FIG. 14, as in FIG. 13, three wires or filars 230p, 230a are connected to weld pads 1032 on the sensor 112, and two wire bonds or jumpers 1330 are connected between two of the weld pads 1032 on the sensor 112 and the two proximal terminals 560 of the sensor mount 412. Two distal wires or filars 930f are connected to the distal terminals 570 of the sensor mount 412, to provide electrical connectivity to the flow sensor, located distal of the pressure sensor mount 412.

The sensor mount 412 also includes a core wire lumen 1015 and an optional solder or glue hole 1017 to facilitate attachment of the sensor mount 412 to the core wire, or to a shaping ribbon as described above. Also visible are the core wire lumen 1015, fixed portion 214 of the sensor 112, a diaphragm or sensing element 1012, cantilevered portion 1016 of the sensor 112, and sensor cantilever recess 540.

Thus, electrical connectivity (e.g., for carrying power and signals) can be provided between the wires or filars 230a and the flow sensor 113, without the need for the wires or filars 230a to extend directly from the connection portion 114 to the flow sensor 113 by running alongside the pressure sensor mount 412. Furthermore, the two electrical pathways defined by wires or filars 230a are shared between the pressure sensor 112 and the flow sensor 113, as described above.

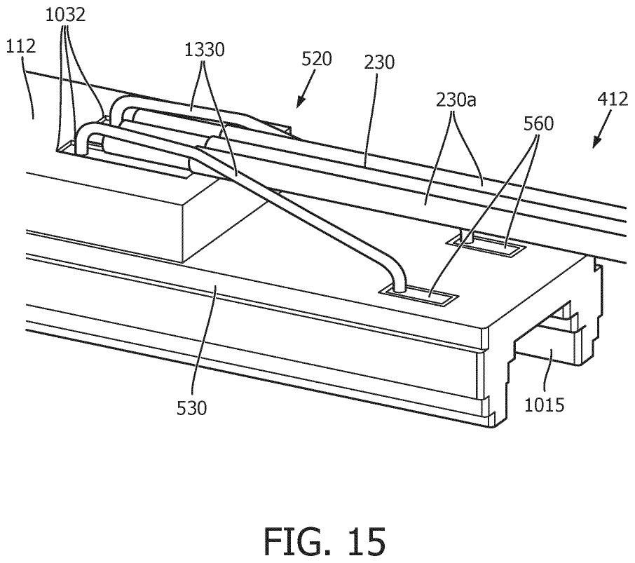

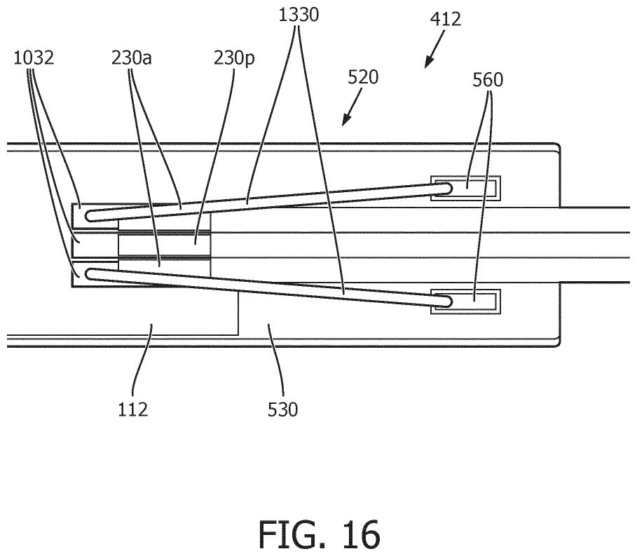

Also visible is a detail region, marked by a dotted rectangle, which will be shown at greater magnification in FIGS. 15 and 16.

FIG. 15 is a diagrammatic, perspective view of the detail region 1490 of the sensor mount 412 of FIG. 14, according to aspects of the present disclosure. Visible are the sensor 112, sensor weld pads 1032, wire bonds or jumpers 1330, wire or filar 230p, shared wires or filars 230a, proximal terminals 560, and core wire lumen 1015.

FIG. 16 is a diagrammatic top view of the detail region 1490 of the sensor mount 412 of FIG. 14, according to aspects of the present disclosure. Visible are the sensor 112, sensor weld pads 1032, wire bonds or jumpers 1330, wire or filar 230p, shared wires or filars 230a, and proximal terminals 560.

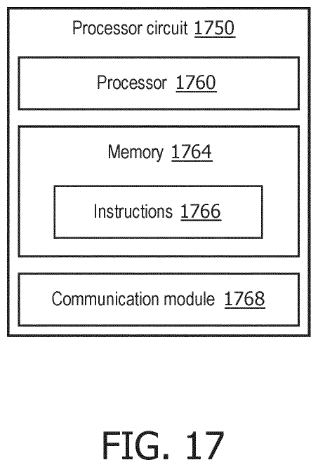

FIG. 17 is a schematic diagram of a processor circuit 1750, according to aspects of the present disclosure. The processor circuit 1750 may be implemented in the intravascular sensing system 100 (e.g., the PIM 304, processing system 306) or other devices or workstations (e.g., third-party workstations, servers, etc.), or on a cloud processor or other remote processing unit, as necessary to implement the method. As shown, the processor circuit 1750 may include a processor 1760, a memory 1764, and a communication module 1768. These elements may be in direct or indirect communication with each other, for example via one or more buses.

The processor 1760 may include a central processing unit (CPU), a digital signal processor (DSP), an ASIC, a controller, or any combination of general-purpose computing devices, reduced instruction set computing (RISC) devices, application-specific integrated circuits (ASICs), field programmable gate arrays (FPGAs), or other related logic devices, including mechanical and quantum computers. The processor 1760 may also comprise another hardware device, a firmware device, or any combination thereof configured to perform the operations described herein. The processor 1760 may also be implemented as a combination of computing devices, e.g., a combination of a DSP and a microprocessor, a plurality of microprocessors, one or more microprocessors in conjunction with a DSP core, or any other such configuration.

The memory 1764 may include a cache memory (e.g., a cache memory of the processor 1760), random access memory (RAM), magnetoresistive RAM (MRAM), read-only memory (ROM), programmable read-only memory (PROM), erasable programmable read only memory (EPROM), electrically erasable programmable read only memory (EEPROM), flash memory, solid state memory device, hard disk drives, other forms of volatile and non-volatile memory, or a combination of different types of memory. In an embodiment, the memory 1764 includes a non-transitory computer-readable medium. The memory 1764 may store instructions 1766. The instructions 1766 may include instructions that, when executed by the processor 1760, cause the processor 1760 to perform the operations described herein. Instructions 1766 may also be referred to as code. The terms “instructions” and “code” should be interpreted broadly to include any type of computer-readable statement(s). For example, the terms “instructions” and “code” may refer to one or more programs, routines, sub-routines, functions, procedures, etc. “Instructions” and “code” may include a single computer-readable statement or many computer-readable statements.

The communication module 1768 can include any electronic circuitry and/or logic circuitry to facilitate direct or indirect communication of data between the processor circuit 1750, and other processors or devices. In that regard, the communication module 1768 can be an input/output (I/O) device. In some instances, the communication module 1768 facilitates direct or indirect communication between various elements of the processor circuit 1750 and/or the intraluminal sensing system 100. The communication module 1768 may communicate within the processor circuit 1750 through numerous methods or protocols. Serial communication protocols may include but are not limited to United States Serial Protocol Interface (US SPI), Inter-Integrated Circuit (I2C), Recommended Standard 232 (RS-232), RS-485, Controller Area Network (CAN), Ethernet, Aeronautical Radio, Incorporated 429 (ARINC 429), MODBUS, Military Standard 1553 (MIL-STD-1553), or any other suitable method or protocol. Parallel protocols may include but are not limited to Industry Standard Architecture (ISA), Advanced Technology Attachment (ATA), Small Computer System Interface (SCSI), Peripheral Component Interconnect (PCI), Institute of Electrical and Electronics Engineers 488 (IEEE-488), IEEE-1284, and other suitable protocols. Where appropriate, serial and parallel communications may be bridged by a Universal Asynchronous Receiver Transmitter (UART), Universal Synchronous Receiver Transmitter (USART), or other appropriate subsystem.

External communication (including but not limited to software updates, firmware updates, or readings from the intraluminal device) may be accomplished using any suitable wireless or wired communication technology, such as a cable interface such as a universal serial bus (USB), micro USB, Lightning, or FireWire interface, Bluetooth, Wi-Fi, ZigBee, Li-Fi, or cellular data connections such as 2G/GSM (global system for mobiles), 3G/UMTS (universal mobile telecommunications system), 4G, long term evolution (LTE), WiMax, or 5G. For example, a Bluetooth Low Energy (BLE) radio can be used to establish connectivity with a cloud service, for transmission of data, and for receipt of software patches. The controller may be configured to communicate with a remote server, or a local device such as a laptop, tablet, or handheld device, or may include a display capable of showing status variables and other information. Information may also be transferred on physical media such as a USB flash drive or memory stick.

Accordingly, it may be seen that the sensor mount with embedded conductors advantageously enables the pass-through of electrical signals from filars that are proximal of the sensor mount to filars or other conductors that are distal of the sensor mount, without the normally routine need to route filars adjacent to the sensor mount. Thus, for a combination intraluminal physiology sensing device that incorporates more than one sensor, the sensor mount for one of the sensors can includes conductive materials that carry signals for a different sensor. Such an arrangement may simplify intraluminal device designs, manufacturing, and assembly procedures, may reduce the chance of manufacturing defects, may reduce costs, may reduce device diameters, and may help make the intraluminal sensing device more robust during handling and use.

The logical operations making up the aspect of the technology described herein are referred to variously as operations, steps, objects, elements, components, or modules. Furthermore, it should be understood that these may be arranged or performed in any order, unless explicitly claimed otherwise or a specific order is inherently necessitated by the claim language. It should further be understood that the described technology may be employed in single-use and multi-use electrical and electronic devices for medical or nonmedical use.

All directional references e.g., upper, lower, inner, outer, upward, downward, left, right, lateral, front, back, top, bottom, above, below, vertical, horizontal, clockwise, counterclockwise, proximal, and distal are only used for identification purposes to aid the reader's understanding of the claimed subject matter, and do not create limitations, particularly as to the position, orientation, or use of the metal ink conductor assembly. Connection references, e.g., attached, coupled, connected, and joined are to be construed broadly and may include intermediate members between a collection of elements and relative movement between elements unless otherwise indicated. As such, connection references do not necessarily imply that two elements are directly connected and in fixed relation to each other. The term “or” shall be interpreted to mean “and/or” rather than “exclusive or.” The word “comprising” does not exclude other elements or steps, and the indefinite article “a” or “an” does not exclude a plurality. Unless otherwise noted in the claims, stated values shall be interpreted as illustrative only and shall not be taken to be limiting.

The above specification, examples and data provide a complete description of the structure and use of exemplary aspects of the metal ink conductor assembly as defined in the claims. Although various aspects of the claimed subject matter have been described above with a certain degree of particularity, or with reference to one or more individual aspects, those skilled in the art could make numerous alterations to the disclosed aspects without departing from the spirit or scope of the claimed subject matter.

Still other aspects are contemplated. It is intended that all matter contained in the above description and shown in the accompanying drawings shall be interpreted as illustrative only of particular aspects and not limiting. Changes in detail or structure may be made without departing from the basic elements of the subject matter as defined in the following claims.

What is claimed is:

1. An intraluminal device, comprising:
a flexible elongate member configured to be positioned within a body lumen of a patient, wherein the flexible elongate member comprises a proximal portion and a distal portion;
a first sensor positioned at the distal portion of the flexible elongate member;
a second sensor positioned at the distal portion of the flexible elongate member; and
a sensor mount positioned at the distal portion of the flexible elongate member,
wherein the first sensor is positioned on the sensor mount,
wherein the second sensor is spaced from the first sensor, and
wherein the sensor mount comprises a first material that is electrically conductive and configured to carry electrical signals associated with the second sensor.

2. The intraluminal device of claim 1,
wherein the first sensor comprises a first intraluminal modality, and
wherein the second sensor comprises a different, second intraluminal modality.

3. The intraluminal device of claim 2,
wherein the first sensor comprises a pressure sensor, and
wherein the second sensor comprises a flow sensor.

4. The intraluminal device of claim 1,
wherein the sensor mount comprises a second material forming an outer surface of the sensor mount,
wherein, in a cross-section, the second material completely surrounds the conductive first material.

5. The intraluminal device of claim 4,
wherein the second material is electrically conductive,
wherein the sensor mount comprises a third material disposed between the first material and the second material,
wherein the third material comprises an electrically insulating material.

6. The intraluminal device of claim 5, wherein, in the cross-section, the third material completely surrounds the first material.

7. The intraluminal device of claim 1,
wherein the sensor mount comprises a proximal portion and a distal portion,
wherein the first material extends between the proximal portion and the distal portion.

8. The intraluminal device of claim 7, wherein the first sensor overlaps with the first material along a length of the sensor mount.

9. The intraluminal device of claim 1,
wherein a majority of the first material is embedded within the sensor mount, and
wherein the first material comprises a first exposed portion and a second exposed portion.

10. The intraluminal device of claim 9, further comprising:
a connector region positioned at the proximal portion of the flexible elongate member;
a first electrical wire coupled to the first exposed portion and the second sensor; and
a second electrical wire coupled to the second exposed portion and the connector region such that the second sensor is in electrical communication with the connector region.

11. The intraluminal device of claim 12, further comprising a third electrical wire coupled to the first sensor and the connector region such that the first sensor is in electrical communication with the connector region.

12. The intraluminal device of claim 9, further comprising:
a connector region positioned at the proximal portion of the flexible elongate member;
a first electrical wire coupled to the first exposed portion and the second sensor;
a wire bond coupled to the second exposed portion and the first sensor; and
a second electrical wire coupled to the first sensor and the connector region such that the first sensor and the second sensor is in electrical communication with the connector region.

13. The intraluminal device of claim 9, wherein the first exposed portion and the second exposed portion are continuous with an outer surface of the sensor mount.

14. An apparatus, comprising:
an intravascular guidewire configured to be positioned within a blood vessel of a patient;
a flow sensor positioned at a distal end of the intravascular guidewire;
a pressure sensor positioned proximal of the flow sensor such that the pressure sensor is spaced from the distal end of the intravascular guidewire;
a pressure sensor mount, wherein the pressure sensor is positioned on the pressure sensor mount;
a connector region positioned at a proximal portion of the intravascular guidewire; and
a flow signal pathway extending between the flow sensor and the connector region, wherein the flow signal pathway is configured to carry electrical signals associated with the flow sensor, wherein a portion of the flow signal pathway comprises conductive material forming part of a structure of the pressure sensor mount.

15. An apparatus of claim 14, further comprising:
a pressure signal pathway extending between the pressure sensor and the connector region, wherein the pressure signal pathway is configured to carry electrical signals associated with the pressure sensor.
